(19) United States
(12) Patent Application Publication
(10) Pub. No.: US 2026/0007532 A1
(43) Pub. Date: Jan. 8, 2026

(54) METHOD OF FIXATION OF INTRALUMINAL DEVICE, INTRALUMINAL FIXATION SYSTEM AND INTRALUMINAL FASTENER

(71) Applicant: BFKW, LLC, Grand Rapids, MI (US)

(72) Inventors: Randal S. Baker, Grand Rapids, MI (US); Frederick J. Walburn, Grand Rapids, MI (US)

(21) Appl. No.: 18/880,338

(22) PCT Filed: Jun. 4, 2024
(86) PCT No.: PCT/IB2024/055456

§ 371 Date: Dec. 31, 2024

Related U.S. Application Data
(60) Provisional application No. 63/506,260, filed on Jun. 5, 2023.
(60) Provisional application No. 63/549,706, filed on Feb. 5, 2024.

(51) Int. Cl. A61F 5/00; A61B 1/273; A61B 17/00; A61B 17/34

(52) U.S. Cl. CPC A61F 5/0036; A61B 1/2736; A61B 17/00234; A61B 17/34; A61F 5/0089; A61B 2017/00296; A61B 2017/00477; A61B 2017/00862; A61B 2017/00867; A61B 2017/00876

(57) ABSTRACT

A method of fixation of an intraluminal device to an inner surface of a hollow organ, an intraluminal fixation system and intraluminal fastener includes positioning a distal end portion of a shaft guide adjacent the outer surface of the hollow organ. A contact distribution member is formed by feeding at least the distal first portion of a flexible shaft through the shaft guide out the distal end portion of the shaft guide. The first portion of the flexible shaft has elastic properties that allow it to be collapsed to fit within the shaft guide and self-expand to a first memory-induced configuration when deployed from the distal end portion of the shaft guide outside of the hollow organ. A biasing member is biased toward the contact distribution member thereby biasing the intraluminal device toward the hollow organ wall.

### BACKGROUND OF THE INVENTION

The present invention is directed to a technique for fastening an intraluminal device, such as a bariatric device or a metabolic treatment device, to the inner surface of an organ such as the gastro-intestinal tract and, in particular, to the cardiac portion of the stomach. The invention is especially useful in the presence of peristalsis which increases the difficulty of sustained fixation.

### SUMMARY OF THE INVENTION

A method of fixation of an intraluminal device to an inner surface of a hollow organ, an intraluminal fixation system and intraluminal fastener, according to an aspect of the invention, includes positioning a distal end portion of a shaft guide adjacent the outer surface of the hollow organ. A contact distribution member that is adapted to distribute physical contact across an enlarged area of an outer surface of the hollow organ wall is formed by feeding at least the distal first portion of a flexible shaft through the shaft guide out the distal end portion of the shaft guide. The first portion of the flexible shaft has elastic properties that allow it to be collapsed to fit within the shaft guide and self-expand to a first memory-induced configuration when deployed from the distal end portion of the shaft guide outside of the hollow organ. A biasing member against a wall of the intraluminal device. A biasing member is biased toward the contact distribution member thereby biasing the intraluminal device toward the hollow organ wall.

The distal first end portion of the shaft guide may be positioned within the hollow organ and the distal end portion of the flexible shaft fed through the organ wall. The biasing member may be biased toward the contact distribution member by being joined to the contact distribution member at the organ wall. The distal end portion of the flexible shaft may be fed through the organ wall including guiding the distal end portion of the shaft guide to a desired location within the hollow organ. The distal end of the shaft guide may be guided with a steerable endoscope.

The contact distribution member may form an open pattern with uncovered spaces between adjacent portion of the flexible shaft. The open pattern may be a floral pattern, a circular pattern, a spiral coil pattern, or similar pattern.

The biasing member may be defined by a proximal second portion of the flexible shaft extending from a distal portion of the first portion. The second portion of the flexible shaft may have elastic properties that allow it to be collapsed to fit within the shaft guide and self-expand to a memory-induced configuration when deployed from the distal end portion of the shaft guide adjacent the intraluminal device. The hollow organ wall may be penetrated at the desired location with a needle point at the distal end of the shaft guide or flexible shaft.

The first and second memory-induced configurations may jointly define a spiral pattern, with the first memory-induced configuration defining outer turns of the spiral pattern and the second memory-induced configuration defining inner turns of the spiral pattern. The biasing member may be separate from the flexible shaft. The biasing member may be a T-shaped fastener. The biasing member may be connected with the contact distribution pattern by a non-metallic filament.

The intraluminal device may be made up a plurality of patches distributed across the inner surface of the hollow organ wall, each patch defining a portion of the intraluminal device. A plurality of fasteners are provided, each with one patch.

The biasing member may be biased toward the contact distribution member by one of the contact distribution member or the biasing member defining a magnet and the other of the contact distribution member or biasing member being magnetically attracted to the magnet.

The amount of stress applied by the intraluminal device to the inner surface of the hollow organ wall may be made adjustable. The flexible shaft may be made from (i) a metal, (ii) a polymer, and/or (iii) a carbon fiber material. The shaft guide may be a hollow opening through a tube or a conduit connected to the outside of the tube.

Embodiments of the invention may be used to fix an intraluminal device having a baroreceptor stimulating surface to the cardiac portion of the stomach in order to stimulate baroreceptors. The intraluminal device may be trans-oral positioned to the stomach. The distal end portion of the shaft guide may be positioned adjacent the outer surface of the stomach percutaneously or laparoscopically to the abdominal cavity or trans-orally through the stomach wall. The intraluminal device may be explanted by removing the flexible shaft including grasping a distal end portion of the flexible shaft and retracting the flexible shaft into a removal conduit.

An intraluminal fastener that is adapted to fix an intraluminal device to the inner surface of a hollow organ and be deployed to from within the hollow organ, according to an aspect of the invention, includes a flexible shaft having a distal end portion and a proximal end portion joined with the distal end portion with a tissue penetrating portion. The distal end portion of the flexible shaft has elastic properties that allow it to be collapsed to fit within a shaft guide and self-expand to a first memory-induced configuration when deployed from the distal end portion of the shaft guide outside of the hollow organ, the first memory-induced configuration defining a contact distribution member that is adapted to distribute physical contact across an enlarged area of an outer surface of the hollow organ wall. The proximal end portion of the flexible shaft has elastic properties that allow it to be collapsed to fit within the shaft guide and self-expand to a second memory-induced configuration when deployed from the distal end portion of the shaft guide adjacent the intraluminal device to bias the intraluminal device against the inner wall of the hollow organ. The tissue penetrating portion is adapted to pass through the hollow organ wall and bias the proximal end portion in the second memory-induced configuration toward the distal end portion in the first memory-induced configuration.

Secure fixation is especially difficult in the presence of peristalsis as occurs in the gastro-intestinal tract. The present invention provides an intraluminal fixation technique that both provides secure fixation of an intraluminal device and is removable to explant the device. The technique may be carried out trans-orally both for implanting and explanting the intraluminal device. Secure fixation is carried out while reducing infection risk to tissue, which is especially important for long-term fixation, by distributing the fixation pressure over a large area of the organ wall with an open, uncovered pattern. Such uncovered pattern allows blood perfusion to the subjacent tissue. Also, there are no surfaces with large surface area or porous surfaces that tend to promote pathogen growth.

Also, the invention is compatible with adjustable intraluminal devices that are adapted to apply variable stress to the organ wall. Embodiments of the invention may be carried out with manual instruments that are manipulated by a human operator, such as a surgeon or other medical person, or may be carried out with end-of-arm tools that are manipulated by a surgical robot of the type that is commercially available from multiple sources.

These and other objects, advantages, purposes and features of this invention will become apparent upon review of the following specification in conjunction with the drawings.

### BRIEF DESCRIPTION OF THE DRAWINGS

FIG. 1 is a perspective view of an intraluminal device deployed to the cardiac portion of the stomach;

FIG. 2 is a plan view of an intraluminal device and method of fixation, according to an embodiment of the invention;

FIG. 3 is a sectional view taken along lines III-III in FIG. 2;

FIG. 4 is a plan view of a fastener according to an embodiment of the invention;

FIG. 5 is a side view of a retractable needle system, according to an embodiment of the invention;

FIG. 6 is a perspective view of an alternative embodiment of an intraluminal device deployed to the cardiac portion of the stomach;

FIG. 7 is an enlarged view of the intraluminal device in FIG. 6 showing the individual patches in various orientations to illustrate details thereof; and

FIG. 8 is a flow diagram of a method according to an embodiment of the invention.

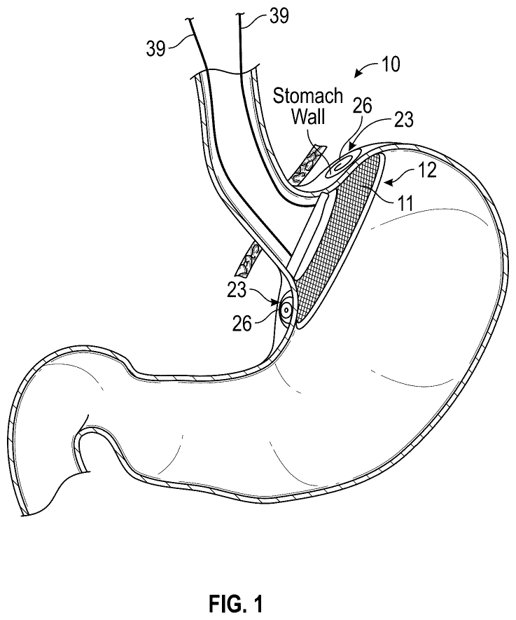

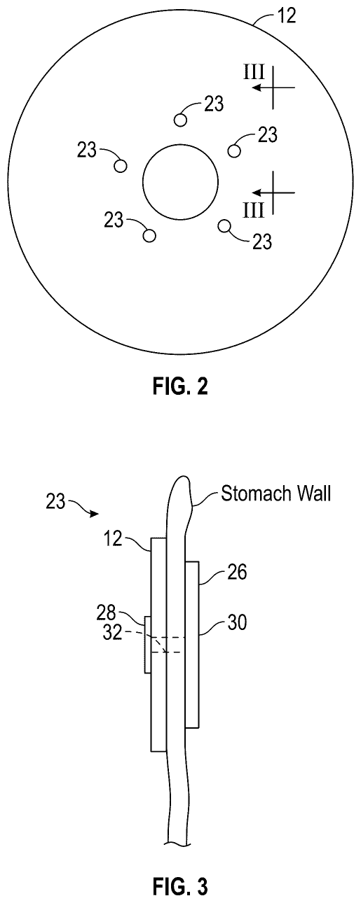

### DESCRIPTION OF THE PREFERRED EMBODIMENTS

The present invention will now be described with reference to the accompanying figures, wherein the numbered elements in the following written description correspond to like-numbered elements in the figures. A method 10 is provided for fixation of an intraluminal device 12 having a body 11 to the inner surface of a hollow organ wall. An example of such a hollow organ is the gastro-intestinal tract and an example of such an inner surface is the cardiac portion of the stomach. In such embodiment of the invention, body 11 defines a baroreceptor stimulating surface facing the hollow organ wall to stimulate baroreceptors on the wall. Other embodiments could alternatively be applied, for example, to position an intraluminal device such a feeding tube or drainage tube to the inner surface of their respective hollow organ, or the like. The GI tract experiences peristalsis which increases the difficulty of affixation of an intraluminal device to the inner surface of the GI tract. Method 10 includes deploying 13 the intraluminal device 12 against the inner surface of the organ where the intraluminal device is fixed to the wall of the hollow organ. The intraluminal device may be deployed to perform one or more functions such as applying stress to the inner surface of the organ. Method 10 includes deploying a shaft guide 18 to intraluminal device 12 in the hollow organ. One or more fasteners 23 include a contact distribution member 26 and a biasing member 28. The contact distribution member is defined by a first self-expanding portion 24 of a flexible shaft 20 that is adapted to distribute physical contact across an enlarged area of an outer surface of the hollow organ wall. The flexible shaft has elastic properties that allow it to be collapsed to fit within shaft guide 18 and self-expanded to a memory-induced configuration when deployed from the distal end-portion of the shaft guide. Biasing member 28, in certain embodiments, extends from a proximal portion 30 of first self-expanding portion 24 of flexible shaft 20 and adapted to bias intraluminal device 12 against the inner surface of the hollow organ wall.

An intraluminal fixation system 14 includes a shaft guide 18 defined as a hollow through-opening formed through tube 16. Alternatively, shaft guide 18 may be formed by a separate conduit attached to tube 16. In the illustrated embodiment, a needle point 19 is formed at a distal end portion of shaft guide 18. Alternatively, needle point 19 may be formed at a distal end of flexible shaft 20 so that the flexible shaft forms its own penetration of the stomach wall at it is fed from a distal-end portion of shaft guide 18. Other alternatives, such as having its own dedicated instrument may be used to define needle point 19. Needle point 19 is positioned within the hollow organ and may be directed within the hollow organ to penetrate the hollow organ wall at a particular location at the intraluminal device by the use of a steering device 22. Steering device 22 may be a steerable endoscope or a dedicated device that can be directed in multiple axes from external the patient to direct the placement of the distal end portion of shaft guide 18 at a desired location at intraluminal device 12. Alternatively, steering device 22 may be a guide wire inserted trans-orally and leading to the intraluminal device in the stomach.

Fastener 23 has a contact distribution pattern 26 and a biasing member 28. The contact distribution pattern distributes stress across a wider area of the tissue defining the outer surface of the hollow organ wall as will be described in more detail. Biasing member 28 is joined to contact distribution pattern 26 through the stomach wall and biases intraluminal device 12 into contact with the inner surface of the hollow organ.

In the illustrated embodiment, a flexible shaft 20 is fed through shaft guide 18 and through the needle point 19 which has penetrated the organ wall. A distal first flexible portion of shaft 24 is fed from the distal end of shaft guide 18 to a space external the organ adjacent the outer wall thereof. The flexible shaft has elastic properties that allow it to be collapsed to fit within shaft guide 18 and self-expanded to a memory-induced configuration when deployed from the distal end portion of the shaft guide. First self-expanding portion 24 of the flexible shaft, when fed through distal needle point 19 and through the hollow organ wall, where first self-expanding portion expands to the memory-induced configuration outside of the hollow organ wall. The memory-induced configuration of flexible shaft 20 first self-expanding portion 24 forms a contact distribution pattern 26 adjacent the outer surface of the hollow organ.

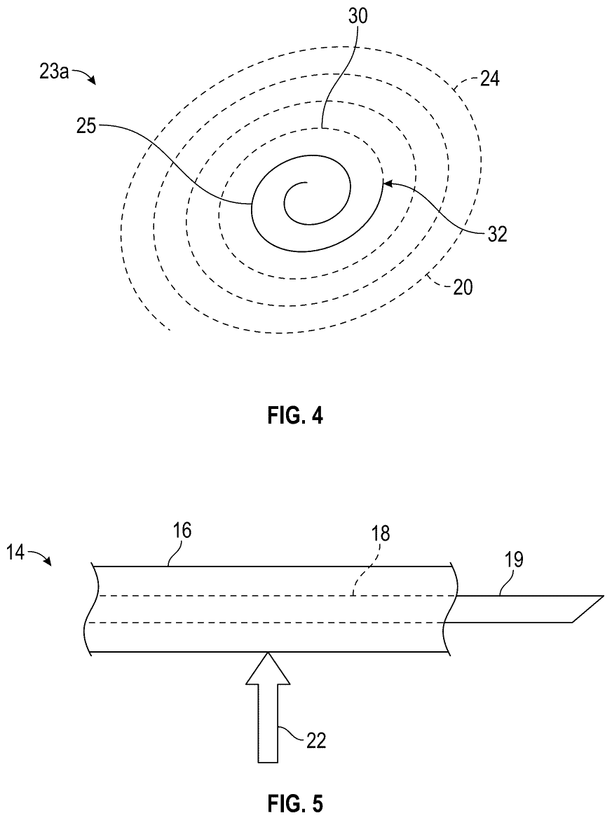

Referring to FIGS. 3 and 4, biasing member 28 extends from a proximal end portion 30 of first self-expanding portion 24 of flexible shaft 20 and has a portion in contact with the intraluminal device. With fastener 23 appropriately dimensioned for the application, as would be within the skill of the average artisan, biasing member 28 sandwiches intraluminal device 12 and the stomach wall against the contact distribution pattern. In this manner, biasing member 28 biases the baroreceptor stimulating surface of body 11 against the inner surface of the stomach.

Contact distribution pattern 26 is an uncovered open pattern formed by the flexible shaft 20. While individual wire segments may be coated for bio-compatibility purposes, there is no overall cover that hides the wire turns in the contact distribution pattern. Such open pattern reduces tissue erosion because there is no area of tissue of the stomach that compressed against the whole contact distribution pattern. Also, wire surface is smooth thus minimizing necrosis. Such open pattern may be a floral pattern, a circular pattern, a spiral coil pattern or the like. The contact distribution pattern 26 is determined by the treatment of the material forming flexible shaft 20 so that it forms the desired pattern upon discharge from the distal end portion of shaft guide 18. Such treatment of flexible shaft 20 to form a desired pattern when in a self-expanded form is known in the art. An example of such a treatment used to manufacture a sealing device for repair of a cardiac defect is disclosed in U.S. Pat. No. 11,589,853, the disclosure of which is hereby incorporated herein by reference in its entirety. Flexible shaft 20 may be made from a material having elastic properties, such as metal wire, such as shape memory nitinol alloy wire or a super-elastic nitinol wire. The elastic wire may be of drawn filled with wire containing a different metal at the core such as a radiopaque metal at the center. Upon deployment, the wire structure resumes its deployed shape without permanent deformation. Alternatively, flexible shaft 20 may be made from other known materials such as a polymer, a carbon fiber, or the like having such memory characteristics.

Intraluminal device 12 may be removed, or explanted, such as after use. This may be accomplished by retracting the first self-expanding portion into shaft guide 16, or similar tube. Advantageously, explanting, as well as deployment, can be accomplished from within the hollow organ, such as the stomach. The interior of the hollow organ is often connected with the outside by an orifice, such as the mouth or anus. Explanting may be accomplished by grasping, such as with an endoscopic grasper, a proximal end portion 30 of the first self-expanding portion 24, which is at penetrating portion 32, and retracting the flexible shaft through the wall of the stomach and into a conduit, such as the working channel of a steerable endoscope, or the like.

Intraluminal device 12 may be deployed to the stomach by trans-oral deployment of the intraluminal device using a conventional endoscope, such as disclosed in commonly assigned International Patent Cooperation Treaty application publication No. WO 2023/166472 entitled Method of Deployment of Intraluminal Device and Intraluminal System, the disclosure of which is hereby incorporated herein by reference in its entirety. Fixation system 15 may be deployed trans-orally to the stomach and guided to the location of the intraluminal device such as with a steerable endoscope. Thus, it is possible to use the same endoscope to both deploy the intraluminal device to the stomach and to guide needle point 19 to position. Alternatively, fixation system 15 may be positioned percutaneously, such as guided by a light source in the stomach. Yet further, fixation system 15 could be deployed from the abdominal cavity by positioning the fixation system 15 laparoscopically to the abdominal cavity.

Flexible shaft 20 may also have a second self-expanding portion 25 that is proximal of first self-expanding portion 24 and which forms biasing member 28. First and second portions 24, 25 are separated by a penetrating portion 32 that is positioned where flexible shaft 20 penetrates the organ wall. Second portion 25 terminates proximally at a proximal end portion 30 which provides an accessible tab that can be grasped, such as with an endoscopic grabber, to be retracted into a conduit, such as the working channel of the endoscope. Proximal end portion 30 may terminate in a soft tip in order to avoid a point of tissue erosion. Unlike first self-expanding portion 24, which contacts human tissue and, therefore is formed as a contact distribution pattern, second self-expanding portion 25 contacts intraluminal device 12 which is more resistant to tear and erosion than human tissue. Therefore, biasing member 28 can have a significantly smaller overall area than contact distribution pattern 26.

An example of a fastener 23a made entirely from the flexible shaft 20a is illustrated in FIG. 4. Flexible shaft 20a is formed as a continuous spiral that defines first self-expanding portion 24a and second self-expanding portion 25b that are separated by a penetrating portion 32a. As flexible shaft 20a is fed distally from shaft guide 18a, first self-expanding portion 24a is discharged from needle point through the stomach wall to outside of the stomach in the abdominal cavity. The largest diameter loops of the spiral defining self-expanding portion 24a form contact distribution pattern 26a. After the length of flexible shaft 20a needed to form contact distribution pattern 26a has been discharged, needle point 19a is withdrawn from the stomach wall and the penetrating portion is positioned at the stomach wall. The remainder of flexible shaft 20a is discharged in the stomach against intraluminal device 12 thereby defining biasing member 28.

While fastener 23a utilizes a continuous spiral to define both first and second self-expanding portions, other shapes will suggest themselves to the skilled artisan. Alternatively, biasing member 28 may be made separately from contact distribution pattern 26 such as from a separate material. For example, biasing member 28 may be formed with a polymeric T-shaped fastener connected with contact distribution pattern 26 by a non-metal filament at the hollow organ wall. Alternatively, first and second self-expanding portions may be made of the same material, such as Nitinol, yet separated by a different material, such as a non-metal, at the penetrating portion 32. Such other material may be selected, for example, for its properties in creating a barrier at the organ wall in order to resist transport of infection from outside of the organ to the interior of the organ or vice versa. An agent such as an antibiotic may be added to the barrier to resist infection. Other shapes and materials for a separated biasing member 28 will be apparent to the skilled artisan.

Alternatively, in other embodiments the intraluminal device may be no larger than required to engage with the biasing member 28, such as a narrow ring shape or the like.

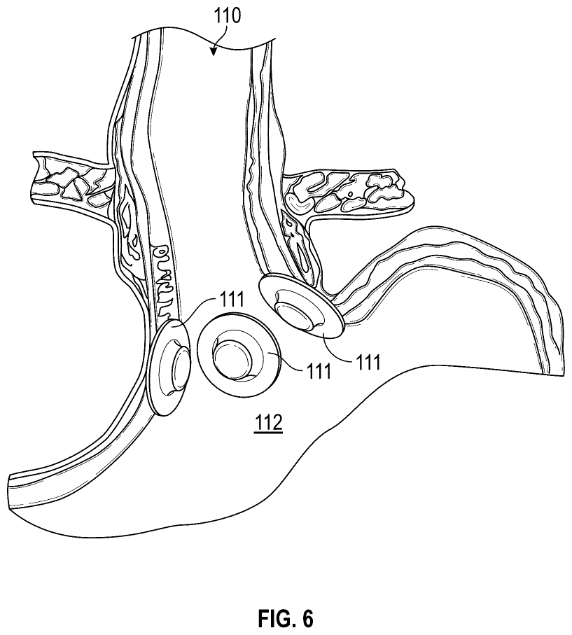

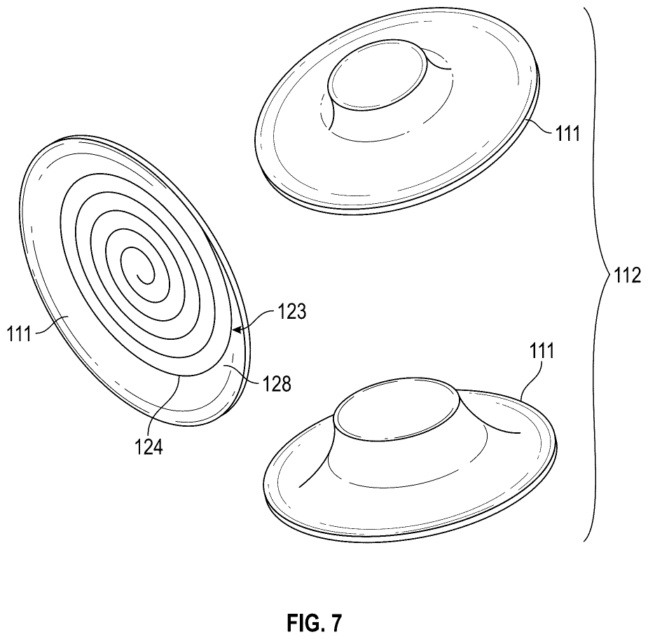

In an alternative embodiment illustrated in FIGS. 6 and 7, an intraluminal device 112 may be defined by a plurality of individual patches 111 each having a separate fastener 123. The individual patches may each define its own biasing member 128 which engages with the contact distribution portion 124 to form each fastener 123. The biasing member may be formed integrally with patch or connected thereto in situ. The individual patches may be distributed across the cardiac portion of the stomach around the GE junction and collectively define a baroreceptor stimulating surface made up of the plurality of separate patches. Such configuration may provide a particular level of stimulus of the baroreceptors in the cardiac portion of the stomach which may be desired for a particular patient characteristic and the level of stimulus may be varied by changing the number of individual patches deployed to the stomach.

The contact distribution portion may be magnetized such as being made from nitinol or a polymer, either having a magnetized core or layer. In such embodiment the biasing member may be made from a ferrous metal incorporated into or attached to the patch and thereby be magnetically attracted to the contact distribution member. Alternatively the biasing member may be magnetized and magnetically attracted to the contact distribution member which is made of a ferrous metal.

The intraluminal device may be used as a bariatric device. The intraluminal device may be used to treat a metabolic disease. The intraluminal device tends to return the upper region of the stomach to its anatomically correct configuration. This tends to resist upward movement to the stomach wall and may be used to treat gastric reflux disease or hiatal hernia.

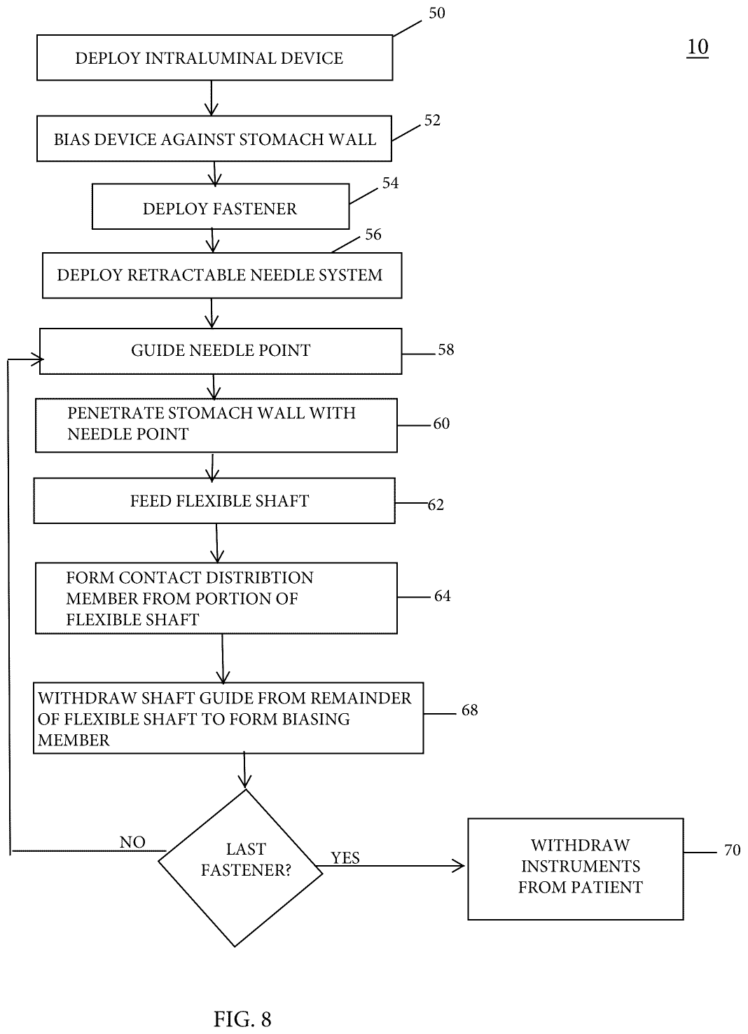

Method of fixation 10 begins by trans-oral deployment 50 of intraluminal device 12 to the interior of the hollow organ, such as to the cardiac portion of the stomach (FIG. 8). This may be accomplished using various techniques, such as the process disclosed in commonly assigned International Patent Cooperation Treaty application publication No. WO 2023/166472 entitled Method of Deployment of Intraluminal Device and Intraluminal System, the disclosure of which is hereby incorporated herein by reference in its entirety. With intraluminal device 12 biased 52 against the cardiac portion of the stomach using retention filament(s) 39 extending from the device through the esophagus to external the patient. Each of a plurality of fasteners 23 are deployed 54 to fix intraluminal device 12 to the cardiac portion of the stomach using the following process, using fastener location parameters set forth in the WO '472 application publication.

Retractable needle system 14 is deployed 56 trans-orally to the stomach. A steering device 22, such as a steerable endoscope, engages a distal end portion of shaft guide 18 and directs 58 needle point 19 into engagement with a location on intraluminal device 12 where a point of fixation is desired. The steering device is guided using direct visualization such as with an endoscope or indirect visualization such as with fluoroscopy, ultrasound or the like. Needle point 19 is inserted 60 through the wall of intraluminal device 12 and through the underlying stomach wall. Alternatively, retractable needle system 14 may be guided to position over a guide wire that is inserted to the patient using known techniques. Complete penetration of the stomach wall by needle point 19 can be confirmed by feeding a fluoroscopic die through needle point 19 and visualizing the die with fluoroscopy. The ensures that the flexible shaft will be fed to the abdominal cavity outside of the stomach and not in the stomach wall.

Flexible shaft 20 is fed 62 through shaft guide 18 to the abdominal cavity where the shaft forms 64 contact distribution pattern 26 as it discharges from the shaft guide. The shaft is fed until penetration portion 32 reaches needle point 19, at which point the shaft guide is withdrawn 66 from the shaft which positions penetration portion 32 at the wall of the stomach. The point of penetration may be treated with an agent, such as a fibrous material, sealing material, antibiotic or the like, to reinforce and seal the tissue and inhibit bacterial growth.

Biasing member 28 is then formed 68 by further feeding of flexible shaft 20 until a second flexible portion 25, which is proximal of first flexible portion 24, is discharged from the shaft guide in the stomach against the outer surface of intraluminal device 12 facing away from the baroreceptor stimulating surface. When the second flexible portion 25 of shaft 20 is discharged from the distal end of the shaft guide, it assumes its memory-induced configuration The memory-induced configuration of second self-expanding portion 25 of flexible shaft 20 defines biasing member 28 and biases the intraluminal device against the cardiac portion of the stomach. When flexible shaft 20 is fully discharged, retractable needle system 14 is withdrawn 70 from the esophagus.

An embodiment of fastener 23a has an expanded shape of flexible shaft 20a in the shape of a spiral pattern with a portion of the spiral pattern defining the first self-expanding portion 24 and a portion of the spiral pattern defining second self-expanding portion 25 (FIG. 4). First self-expanding portion 24 is defined as the larger diameter wraps of the spiral, thereby providing a large surface area of engagement with the outer surface of the stomach in order to reduce erosion of the underlying tissue. The spiral does not have an overall cover (although the flexible shaft may be covered wire) which minimizes infection risk by reducing areas of lack of blood perfusion to tissue, which could promote infection growth. Also, surfaces of flexible shaft 20 are smooth, with minimal surface defects that could also promote infection growth.

Biasing member 28 could alternatively be formed from a non-self-expanding material, such as a polymer, carbon fiber or the like. Such biasing member may be in various forms such as a T-shaped fastener, or the like. Biasing member 28 may be connected with the contact distribution pattern 26 by a non-metal filament at the stomach wall.

Amount of pressure applied by fixation system 15 can be fixed or adjustable. The amount of stress applied by intraluminal device 12 to the cardiac portion of the stomach may be made adjustable using techniques disclosed in detail in commonly assigned U.S. Pat. No. 11,642,234 and patent application publication nos. WO 2020/183399 and US 2026/0151233, the disclosures of which are hereby incorporated herein by reference in their entireties. In addition to varying strength of stress applied to the barro receptors at the cardiac portion of the stomach, varying stress applied by intraluminal device 12 may also be configured to reduce tissue ischemia.

Illustrated in FIG. 11 is a surgical robotic system 400 that is useful in carrying out the invention. Robotic system 400 has an esophageal robotic portion end-of-arm tool 404 that both deploys the stomach assembly to the stomach and visualizes the intraluminal device while it is being fastened. Robotic system 400 further has an abdominal robotic portion 406 that carries out deploying of the abdomen assembly in the abdomen retained in a position around the upper stomach. A robotic control 402 adjusts the relative movement between tools 404 and 406 to ensure that the intraluminal device is properly engaged by the fasteners and that the anatomy of the patient is not compromised by the deployment process. For example, if used with a surgical robot, one robot arm could operate the laparoscopic instrument in the form of an end of arm tool. Another robot arm could operate endoscope 25 as an end of arm tool. Images captured by the endoscope in the stomach could then be used by a common robot controller.

Changes and modifications in the specifically described embodiments can be carried out without departing from the principles of the present invention which is intended to be limited only by the scope of the appended claims, as interpreted according to the principles of patent law including the doctrine of equivalents.

1. A method of fixation of an intraluminal device to an inner surface of a hollow organ through a shaft guide, said method comprising:
positioning a distal end portion of a shaft guide adjacent the outer surface of the hollow organ;
forming a contact distribution member that is adapted to distribute physical contact across an enlarged area of an outer surface of the hollow organ wall, comprising feeding at least the distal first portion of a flexible shaft through said shaft guide out the distal end portion of the shaft guide;
wherein the first portion of the flexible shaft has elastic properties that allow it to be collapsed to fit within the shaft guide and self-expand to a first memory-induced configuration when deployed from the distal end portion of the shaft guide outside of the hollow organ thereby forming the contact distribution member outside of the hollow organ;
positioning a biasing member against a wall of the intraluminal device; and
biasing the biasing member toward the contact distribution member thereby biasing the intraluminal device toward the hollow organ wall.

2. The method as claimed in claim 1 including selectively positioning the distal first end portion of the shaft guide within the hollow organ and feeding the distal end portion of the flexible shaft through the organ wall.

3. The method as claimed in claim 2 wherein said biasing member is biased toward the contact distribution member by being joined to the contact distribution member at the organ wall.

4. The method as claimed in claim 1 wherein said contact distribution member forms an open pattern with uncovered spaces between adjacent portion of the flexible shaft.

5. The method as claimed in claim 2 wherein said open pattern comprises a floral pattern, a circular pattern or a spiral coil pattern.

6. A method of fixation of an intraluminal device to an inner surface of a hollow organ through a shaft guide, said method comprising:
positioning a distal end portion of a shaft guide from within the hollow organ through a wall of the hollow organ to a position adjacent the outer surface of the hollow organ;
forming a contact distribution member that is adapted to distribute physical contact across an enlarged area of an outer surface of the hollow organ wall, comprising feeding at least the distal first portion of a flexible shaft through said shaft guide out the distal end portion of the shaft guide;
wherein the first portion of the flexible shaft has elastic properties that allow it to be collapsed to fit within the shaft guide and self-expand to a first memory-induced configuration when deployed from the distal end portion of the shaft guide outside of the hollow organ;
positioning a biasing member against a wall of the intraluminal device; and
biasing the biasing member toward the contact distribution member thereby biasing the intraluminal device toward the hollow organ wall, wherein said biasing member comprising a proximal second portion of said flexible shaft extending from a distal portion of said first portion of said flexible shaft, wherein the second portion of the flexible shaft has elastic properties that allow it to be collapsed to fit within the shaft guide and self-expand to a memory-induced configuration when deployed from the distal end portion of the shaft guide adjacent the intraluminal device.

7. The method as claimed in claim 6 including feeding the distal end portion of the flexible shaft through the organ wall including guiding the distal end portion of the shaft guide to a desired location within the hollow organ.

8. The method as claimed in claim 7 including penetrating the hollow organ wall at the desired location with a needle point at the distal end of the shaft guide or flexible shaft.

9. The method as claimed in claim 7 wherein said guiding comprises positioning the distal end of said shaft guide with a steerable endoscope.

10. The method as claimed in claim 6 wherein the first and second memory-induced configurations jointly define a spiral pattern, with the first memory-induced configuration defining outer turns of the spiral pattern and the second memory-induced configuration defining inner turns of the spiral pattern.

11. The method as claimed in any of claims 1 wherein said biasing member is separate from said flexible shaft.

12. The method as claimed in claim 11 wherein said biasing member comprises a T-shaped fastener.

13. The method as claimed in claim 11 wherein said biasing member is connected with the contact distribution pattern by a non-metallic filament.

14. The method as claimed in claim 1 wherein intraluminal device comprises at least one patch, said at least one patch defining said biasing member, wherein said at least one fastener comprises a plurality of fasteners and said at least one patch comprises a plurality of patches distributed across the inner surface of the hollow organ wall.

15. The method as claimed in claim 1 wherein biasing the biasing member toward the contact distribution member comprises one of said contact distribution member or said biasing member defining a magnet and the other of said contact distribution member or said biasing member being magnetically attracted to the magnet.

16. The method as claimed in claim 1 including adjusting amount of stress applied by the intraluminal device to the inner surface of the hollow organ wall.

17. The method as claimed in claim 1 wherein said flexible shaft is made from at least one material selected from (i) a metal, (ii) a polymer, and (iii) a carbon fiber material.

18. The method as claimed in claim 1 wherein said shaft guide comprises a hollow opening through a tube or a conduit connected to the outside of said tube.

19. The method as claimed in any of the preceding claims used to fix an intraluminal device having a baroreceptor stimulating surface to the cardiac portion of the stomach in order, to stimulate baroreceptors, wherein said deploying comprises trans-oral positioning of the intraluminal device to the stomach and positioning a distal end portion of a shaft guide adjacent the outer surface of the stomach percutaneously or laparoscopically to the abdominal cavity or trans-orally through the stomach wall.

20. A method of fixation of an intraluminal device to an inner surface of a hollow organ through a shaft guide, said method comprising:
positioning a distal end portion of a shaft guide from within the hollow organ through a wall of the hollow organ to a position adjacent the outer surface of the hollow organ;
forming a contact distribution member that is adapted to distribute physical contact across an enlarged area of an outer surface of the hollow organ wall, comprising feeding at least the distal first portion of a flexible shaft through said shaft guide out the distal end portion of the shaft guide;
wherein the first portion of the flexible shaft has elastic properties that allow it to be collapsed to fit within the shaft guide and self-expand to a first memory-induced configuration when deployed from the distal end portion of the shaft guide outside of the hollow organ;
positioning a biasing member against a wall of the intraluminal device; and
biasing the biasing member toward the contact distribution member thereby biasing the intraluminal device toward the hollow organ wall; and
explanting the intraluminal device by removing the first self-expanding portion comprising grasping a distal end portion of the flexible shaft and retracting the flexible shaft into a removal conduit.

21. An intraluminal fixation system that is adapted to affix an intraluminal device to an inner surface of a hollow organ, comprising:
a shaft guide having a distal end portion;
a steering device adapted to steer the distal end portion of the shaft guide from within the organ to a position adjacent the outer surface of the hollow organ;
a fastener having a contact distribution member that is adapted to distribute physical contact across an enlarged area of an outer surface of the hollow organ wall, wherein the first portion of the flexible shaft has elastic properties that allow it to be collapsed to fit within the shaft guide and self-expand to a first memory-induced configuration when deployed from the distal end portion of the shaft guide outside of the hollow organ thereby forming the contact distribution member;
said fastener further having a biasing member that is biased toward the contact distribution member to bias the intraluminal device against a wall of the hollow organ when the contact distribution member is contacting the outer surface of the hollow organ.

22. An intraluminal fastener that is adapted to fix an intraluminal device to the inner surface of a hollow organ and be deployed to from within the hollow organ, said intraluminal fastener comprising:
a flexible shaft having a distal end portion and a proximal end portion joined with the distal end portion with a tissue penetrating portion;
wherein the distal end portion of the flexible shaft has elastic properties that allow it to be collapsed to fit within a shaft guide and self-expand to a first memory-induced configuration when deployed from the distal end portion of the shaft guide outside of the hollow organ, the first memory-induced configuration defining a contact distribution member that is adapted to distribute physical contact across an enlarged area of an outer surface of the hollow organ wall;
wherein the proximal end portion of the flexible shaft has elastic properties that allow it to be collapsed to fit within the shaft guide and self-expand to a second memory-induced configuration when deployed from the distal end portion of the shaft guide adjacent the intraluminal device to bias the intraluminal device against the inner wall of the hollow organ; and
wherein the tissue penetrating portion is adapted to pass through the hollow organ wall and bias the proximal end portion in the second memory-induced configuration toward the distal end portion in the first memory-induced configuration.
